(19) United States
(12) Patent Application Publication
(10) Pub. No.: US 2014/0100283 A1
(43) Pub. Date: Apr. 10, 2014

(54) Method to Increase Absorption and Bioavailability of Oral Glutathione

(71) Applicant: Atlantic Pro Nutrients, Inc. dba XYMOGEN, Orlando, FL (US)

(72) Inventor: Mike Mahoney, Oklahoma City, OK (US)

(73) Assignee: Atlantic Pro Nutrients, Inc. dba XYMOGEN, Orlando, FL (US)

(21) Appl. No.: 14/047,645

(22) Filed: Oct. 7, 2013

Related U.S. Application Data
(60) Provisional application No. 61/710,515, filed on Oct. 5, 2012.

(51) Int. Cl. A61K 9/48; A61K 31/198

(52) U.S. Cl. CPC A61K 9/4866; A61K 31/198
USPC 514/562

(57) ABSTRACT

An improved method for delivery of glutathione, a major intracellular antioxidant, by oral administration of S-acetyl-glutathione (SAG) is provided. The method is designed to raise the circulating blood levels of glutathione via the administration of an effective amount of SAG in a delayed release composition.

This application claims the benefit of earlier filed U.S. Patent Application Ser. No. 61/710,515, filed on Oct. 5, 2012 and which is hereby incorporated by reference.

### FIELD OF THE INVENTION

An improved method for delivery of glutathione, a major intracellular antioxidant, by oral administration of S-acetyl-glutathione (SAG) is provided. More particularly, the methods disclosed herein are designed to raise the circulating blood levels of glutathione via the administration of SAG in a delayed release composition. Such methods and compositions offer the potential for nutritional intervention in a wide variety of conditions that are associated with reduced glutathione, increased oxidative stress, and/or increased inflammation.

### BACKGROUND

Glutathione, a tri-peptide molecule consisting of the amino acids L-glycine, L-csyteine, and L-glutamic acid (also known as L-glutamate), is the major intracellular antioxidant in humans.

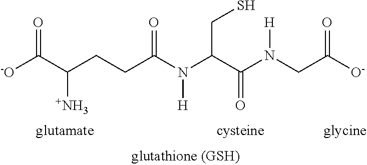

Glutathione exists in reduced (GSH) and oxidized (GSSG) states, with the reduced form (GSH) predominating. An increased GSSG-to-GSH ratio is considered indicative of oxidative stress.

Although glutathione is produced in most cells, many diseases and other pathologies are associated with reduced levels of intracellular glutathione. Glutathione is readily transported across cell membranes, so that increasing circulating glutathione levels in blood would facilitate the intracellular uptake of glutathione and alleviate depleted glutathione levels observed in a variety of disease conditions.

Glutathione is a tightly regulated intracellular constituent, and is limited in its production by negative feedback inhibition of its own synthesis through the enzyme gamma-glutamylcysteine synthetase, thus greatly minimizing any possibility of overdosage. Glutathione augmentation using precursors of glutathione synthesis or intravenous glutathione is a strategy developed to address states of glutathione deficiency, high oxidative stress, immune deficiency, and xenobiotic overload in which glutathione plays a part in the detoxification of the xenobiotic in question (especially through the hepatic route). Glutathione deficiency states include, but are not limited to, HIV/AIDS, chemical and infectious hepatitis, myalgic encephalomyelitis chronic fatigue syndrome, prostate and other cancers, cataracts, Alzheimer's disease, Parkinson's disease, chronic obstructive pulmonary disease, asthma, radiation poisoning, malnutritive states, arduous physical stress, and aging, and has been associated with suboptimal immune response. Many clinical pathologies are associated with oxidative stress and are elaborated upon in numerous medical references.

Traditional methods for delivering glutathione orally are limited by the poor absorption and bioavailability of glutathione itself. It is known that the systemic availability of glutathione when delivered by oral administration is very low; oral administration of a single dose of 3 grams of glutathione to health humans does not increase circulating glutathione to a clinically beneficial extent (Witschi et al., “The Systemic Availability of Oral Glutathione,” Eur. Jour. Clin. Pharmacol., Vol. 43, pp. 667-669, 1992). In addition, the unprotected peptide is subject to degradation in the acid environment of the stomach.

Previous methods for increasing circulating and/or intracellular glutathione levels include: 1) intravenous administration of glutathione; 2) oral administration of glutathione; and 3) administration of glutathione precursors. However, such methods have been either unpractical and/or unsuccessful. For example, methods involving intravenous administration of glutathione are generally not practical for increasing circulating glutathione for most individuals because this procedure is expensive, requires regular visits to a medical practitioner's office, is unsuitable for children and many adults, and poses the risks of side effects associated with regular injections. Oral administration of glutathione is also not practical and may be ineffective since glutathione is not readily absorbed in the gastrointestinal track, as discussed above. Typically, high doses (i.e., 5-10 grams) of glutathione are required to increase glutathione levels. However, ingestion of such high doses of glutathione is often accompanied by undesirable side effects such as nausea and/or other negative effects (e.g., taste aversion). Finally, administration of glutathione precursors such as whey protein, vitamin C, and glutamine, which are often recommended to boost glutathione levels in the body, typically does not raise the level of glutathione by a significant amount, require high doses to be effective, and cost significantly more than an equivalent amount of glutathione. Further, biological individuality is such that not every body has equivalent ability to metabolize the precursor to raise glutathione.

Previous attempts to overcome these problems have either been unsuccessful and/or not feasible. In some cases, these remedies would require substantial cost and labor and might then present new issues such as regulatory issues. This would be the case if the glutathione molecule was substantially modified structurally (i.e., chemically), so that it would no longer be regarded as a dietary supplement by the Food and Drug Administration. There is really no feasible way to reduce the side effects of intravenous administration; these side effects are inherent with intravenous infusion of any substance. Additionally, there is no feasible way to overcome the side effects of oral administration of glutathione, since simply increasing the dose does not result in increased circulating blood levels and increases the frequency of unwanted side effects.

In view of the above, if a way could be found to elevate circulating and intracellular levels of glutathione, this would offer an interventional approach to overcome disease-mediated deficiencies in intracellular glutathione levels.

### SUMMARY

A general object is to provide a method for increasing circulating and/or intracellular levels of glutathione.

A more specific object is to overcome one or more of the problems described above.

In one embodiment, a method for increasing circulating and/or intracellular levels of glutathione includes administering an effective amount of S-acetyl-glutathione (SAG) in the form of delayed-release composition to raise the circulating levels of glutathione to achieve clinically beneficial effects.

In accordance with another embodiment, a method for increasing circulating and/or intracellular levels of glutathione comprises orally administering to an individual in need thereof a delayed-release, gastro-resistant capsule which contains an amount of S-acetyl-glutathione (SAG) effective to increasing circulating and/or intracellular levels of glutathione.

In accordance with a further embodiment, a method for reducing or alleviating one or more symptoms associated with reduced glutathione, increased oxidative stress, and/or increased inflammation includes administering an effective of amount of S-acetyl-glutathione (SAG) in a delayed-release dosage form to an individual in need of such treatment.

### DETAILED DESCRIPTION

One or more of the above objects can be achieved, at least in part, by orally administering to an individual in need thereof an effective amount of a direct precursor of glutathione, S-acetyl-glutathione (SAG), in a delayed release delivery form that is orally bioavailable to achieve a significant elevation in circulating glutathione levels. Such method overcomes the expense and/or inconvenience of intravenous glutathione administration and also overcomes the increased likelihood of side-effects associated with the ingestion of high doses of glutathione. Additionally, significant elevation in circulating glutathione levels can be achieved at a significantly lower cost than any of the existing examples and overcome the regulatory obstacle of structurally modifying glutathione to increase or enhance its bioavailability.

S-acetyl-glutathione (SAG), a lipid-like molecule, is an acetylated form of glutathione wherein the acetyl group is attached to the sulfur atom of cysteine in the glutathione molecule. SAG is one of several reversible bioconjugates of glutathione that are stable in plasma, and that can be taken up by cells directly. De-acetylation is an ordinary occurrence in cells, and once in the cells, SAG is de-acetylated to glutathione to replenish glutathione for mitochondrial action.

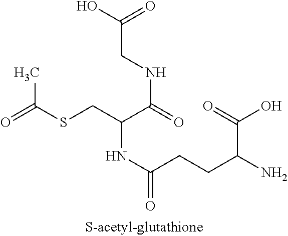 [SMILES: CC(=O)SCC(NC(=O)CCC(N)C(=O)O)C(=O)NCC(=O)O]

In accordance with one embodiment, a method for increasing circulating and/or intracellular levels of glutathione includes administering an effective amount of S-acetyl-glutathione (SAG) in the form of delayed-release composition to raise the circulating levels of glutathione to achieve clinically beneficial effects.

In accordance with another embodiment, S-acetyl-glutathione (SAG) is provided in a delayed release delivery system designed to protect the molecule from degradation by gastric acids in the stomach and carry the SAG to the small intestine where it can be readily taken up intact by chylomicrons in the gut.

Delivering SAG in an acid-resistant, delayed release delivery system provides one or more advantages over previous methods including: a). ease of use; b). convenience; c). more efficient way to raise glutathione; d). achieves higher levels of circulating glutathione; e.) achieve therapeutically effective levels of circulating and/or intracellular glutathione using a lower dosage of SAG compared to oral administration of glutathione and/or other glutathione precursors; f). lower cost will enable more individuals to benefit from this intervention; and g). minimize unpleasant side-effects.

In one aspect, a delayed release delivery form of S-acetyl-glutathione (SAG) can be prepared by encapsulating an amount of SAG within an acid-resistant gel capsule. Gel capsules suitable for use in preparing SAG compositions discloses herein should protect ingredients from full disintegration in the stomach and target capsule disintegration for the small intestine. Additionally, such capsules may mask taste and/or odor in capsule dosage form.

In another aspect, the gel capsule may be formed from hydroxypropyl methylcellulose (HPMC) or hypromellose. One suitable HPMC gel capsule for use in preparing SAG compositions is commercially available under the brand name DRCaps™ from Capsugel, a Belgium company having corporate offices in Peapack, N.J.

In accordance with another embodiment, a method for raising circulating levels of glutathione to achieve clinically beneficial effects includes orally administering one or more doses of delayed-release S-acetyl-glutathione to an individual in need thereof. Such dose may include about 200 to about 1000 milligrams (mg) per day.

In accordance with a further aspect, an effective daily amount of S-acetyl-glutathione (SAG) may be orally administered to an individual in need thereof in one to four doses in the form of 2 to 10 acid-resistant capsules.

In accordance with one embodiment, a method for increasing circulating glutathione levels includes orally administering to an individual in need thereof about 100 to about 400 milligrams of S-acetyl-glutathione (SAG) encapsulated in one or more acid-resistant, delayed-release gel capsules. The SAG may be administered one to four doses per day.

In accordance with a further embodiment, a method for reducing and/or alleviating one or more symptoms associated with reduced glutathione, increased oxidative stress, and/or increased inflammation includes administering an effective of amount of S-acetyl-glutathione (SAG) to an individual in need of such treatment. The effective amount is of SAG may be administered in the form of 2 to 10 delayed-release capsules and provide about 200 to about 1000 mg of SAG per day.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

I claim:

1. A method for increasing circulating and/or intracellular levels of glutathione, comprising:
administering, to an individual in need thereof, an effective amount of S-acetyl-glutathione (SAG) in the form of delayed-release composition;
wherein to the circulating levels of glutathione are increased to achieve clinically beneficial effects.

2. The method according to claim 1, wherein the effective amount of SAG comprises about 200 milligrams (mg) to about 1000 mg on a daily basis.

3. The method according to claim 1, wherein the effective amount of SAG is provided in the form of 2 to 10 acid-resistant gel capsules.

4. The method according to claim 3, wherein the acid-resistant gel capsules comprises hydroxypropyl methylcellulose.

5. The method according to claim 3, wherein the acid-resistant gel capsule protects SAG from full disintegration in the stomach and target capsule disintegration for the small intestine.

6. The method according to claim 1, wherein the clinically beneficial effects comprise one or more benefits selected from the group consisting of reduced glutathione deficiency, reduced levels of oxidative stress, reduced immune deficiency, improved xenobiotic detoxification, and combinations thereof.

7. A method for reducing or alleviating one or more conditions or symptoms associated with glutathione deficiency, increased oxidative stress, and/or increased inflammation, comprising:
orally administering an effective of amount of S-acetyl-glutathione (SAG) to an individual in need of such treatment;
wherein the effective amount of SAG is provided in the form of one or more delayed-release gel capsules.

8. The method according to claim 7, wherein the effective amount comprises about 200 mg to about 1000 mg on a daily basis.

9. The method according to claim 7, wherein the one or more conditions associated with glutathione deficiency is selected from the group consisting of HIV/AIDS, chemical and infectious hepatitis, myalgic encephalomyelitis chronic fatigue syndrome, prostate cancer, cataracts, Alzheimer's disease, Parkinson's disease, chronic obstructive pulmonary disease, asthma, radiation poisoning, malnutritive states, arduous physical stress, aging, and combinations thereof.
